(19) United States
(12) Patent Application Publication
(10) Pub. No.: US 2020/0287704 A1
(43) Pub. Date: Sep. 10, 2020

(54) Color Image Encryption Method Based on DNA Strand Displacement Analog Circuit

(71) Applicants: Qiang Zhang, Dalian City (CN); Chengye Zou, Dalian City (CN); Changjun Zhou, Dalian City (CN); Xiaopeng Wei, Dalian City (CN)

(72) Inventors: Qiang Zhang, Dalian City (CN); Chengye Zou, Dalian City (CN); Changjun Zhou, Dalian City (CN); Xiaopeng Wei, Dalian City (CN)

(21) Appl. No.: 16/881,021

(22) Filed: May 22, 2020

(51) Int. Cl. H04L 9/00; G16B 50/40; H04L 9/08

(52) U.S. Cl. CPC H04L 9/001; H04L 9/0861; G16B 50/40

(57) ABSTRACT

The invention relates to the field of strand displacement, and provides a color image encryption method based on a DNA strand displacement analog circuit. Firstly, a reaction module with a light-emitting group and a quenching group is designed through Visual DSD software, and by utilizing the equivalence of a DNA strand displacement reaction module and an ideal reaction module, an analog circuit formed by the DNA strand displacement reaction module can perform analog on the dynamics characteristics of an ideal reaction network formed by the ideal reaction module, wherein the Rössler chaotic system can be described by the idealized reaction network. Secondly, data generated by the DNA strand displacement analog circuit is converted into a chaotic sequence matched with a plaintext image in size after being extended, and finally, the color image encryption effect is achieved through scrambling and diffusion operations.

### TECHNICAL FIELD

The invention relates to the field of DNA strand displacement, wherein color image encryption is performed by utilizing chaotic sequences generated by a DNA strand displacement analog chaotic circuit.

### BACKGROUND ART

DNA strand displacement is a new technique and method in DNA computing, and takes full advantage of the properties of DNA molecules, for example, the composition of DNA long strands is based on the base complementary pairing principle, that is, the Wastson-Crick complementary pairing principle, so that the DNA strand displacement reaction process becomes a spontaneous reaction, which can be operated at normal temperature without annealing operation or electric field action, and the experimental operation time is short and the product yield is high, so that the DNA strand displacement reaction is more convenient and efficient compared with the traditional molecular assembly. As another example, DNA strands have powerful coding and compiling capabilities, its own DNA sequence can store and compile large amounts of information, one short strand of DNA (20 nt-80 nt) can represent a logic signal, and several strands of DNA can be assembled into a molecule operation gate or logic gate, which makes it easy to construct digital circuits and analog circuits based on DNA strands. DNA strands have great application potential in the field of encryption due to powerful compiling capability.

Unpredictability, ergodicity, pseudorandom and sensitivity to initial value are the main characteristics of chaotic systems, and the main characteristics can be applied to the fields of encryption and secure communication. Therefore, chaotic encryption arouses wide public concern, and people have done a lot of research work on chaotic encryption. However, some image encryption methods based on low-dimensional chaotic systems are not secure, because the low-dimensional chaotic systems often have problems of periodic orbits and lack of secret key space due to the limitation of accuracy in the process of implementation, people turn their attention to high-dimensional chaotic systems and spatiotemporal chaotic systems, but the high-dimensional chaotic systems and spatiotemporal chaotic systems are difficult to achieve. Therefore, how to ensure the security of the encryption scheme is very meaningful in current chaotic image encryption under the condition of limited accuracy.

### SUMMARY OF THE INVENTION

There are a lot of uncertainties and leakage reactions in a DNA strand displacement analog circuit, and the results of the DNA strand displacement analog circuit are difficultly verified by experiments because the reaction rate is difficult to set accurately. At present, most of DNA strand displacement analog circuits are designed and implemented by Visual DSD software. However, the DNA strand displacement analog circuit is a continuous system, can dynamically react to the dynamic behavior of the system, and is conducive to the realization of the dynamic behavior of a chaotic system.

A Rössler chaotic system is subjected to analog by a DSD analog circuit, and the generated chaotic sequence is applied to color image encryption. Compared with the traditional chaotic encryption scheme, this encryption scheme has the following advantages: on the one hand, the secret key is unrelated to the initial value of the chaotic system, but related to the color image itself, so that the space size of the secret key is not affected by the accuracy of the chaotic sequence; on the other hand, due to the fact that the generation of the chaotic sequence relies upon biochemical tests, concentration is measured every 3 seconds to obtain a piece of data, in such a way, a large amount of time is consumed in the process of generating the chaotic sequence. The invention adopts two measures to greatly reduce the time required for measurement, first, the chaotic sequence is divided into blocks, except the first sequence block, data in the rest sequence blocks are the same; and second, a series of secret keys are generated by utilizing the information of plaintext images, and are independent of chaotic sequences, so the same-size plaintext images can utilize the same chaotic sequence, that is to say, there is no need to re-measure the concentration.

Compared with the prior art, the invention has the following advantages:

1. The invention performs color image encryption by utilizing a DNA strand displacement chaotic analog circuit for the first time.
2. The chaotic sequence is divided into blocks, which greatly reduces the number of data to be measured and shortens the measurement time.
3. The secret key is related to the information of the plaintext images and independent of the initial values of the chaotic circuit, so that the encryption effect can still be achieved in the case of low accuracy of chaotic sequences, and the secret key space can resist violence attacks.
4. The secret key is related to the information of the plaintext images and are independent of the initial values of the chaotic circuit. The same-size plaintext images can utilize the same chaotic sequence, so that the detection time can be omitted.

### BRIEF DESCRIPTION OF THE DRAWINGS AND TABLES

FIG. 1 Encryption flow

FIG. 2 Catalytic reaction module 1: X→2X

FIG. 3 Catalytic reaction module 2: X+Y→2Y

FIG. 4 Annihilation reaction module: X+Y→∅

FIG. 5 Degradation reaction module 1: X+X→X

FIG. 6 Degradation reaction module 2: Y→∅

FIG. 7 Experimental results obtained by using encryption and decryption schemes

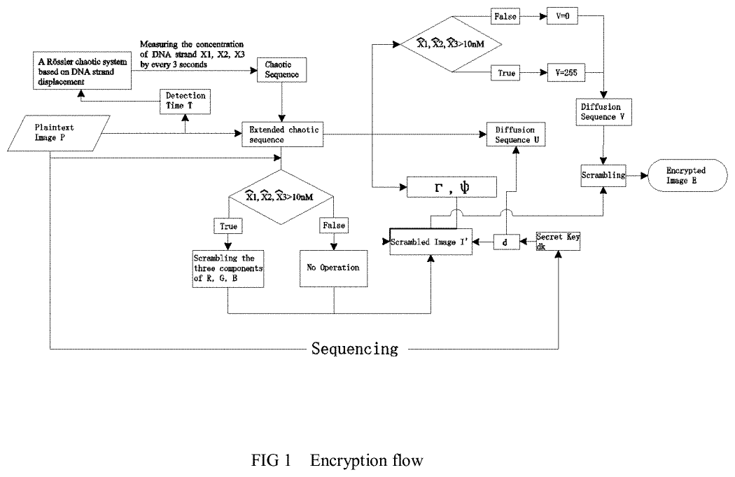

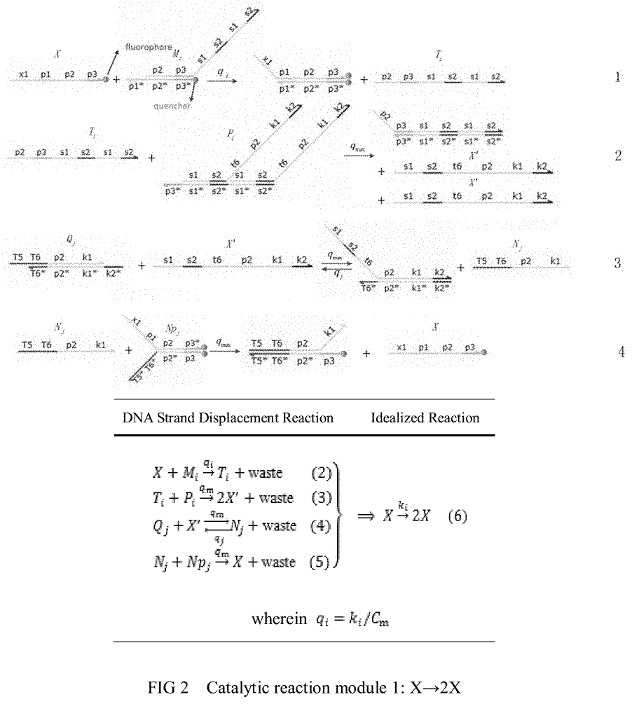

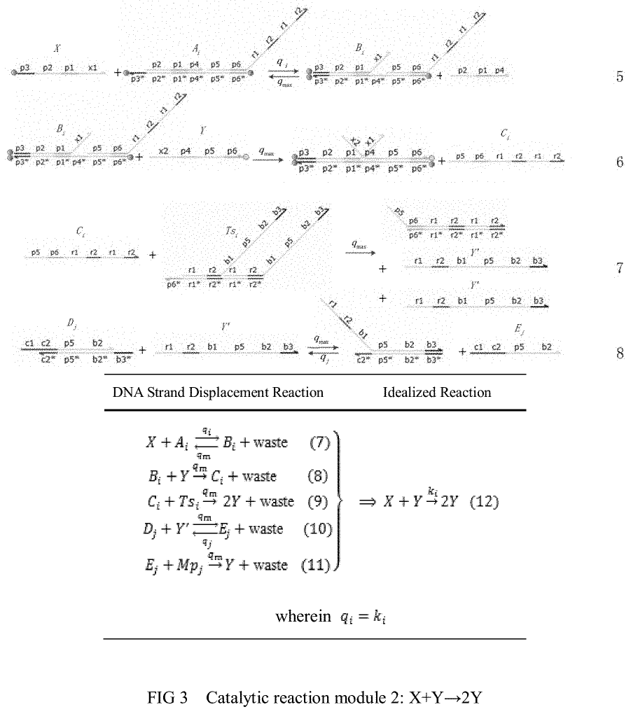

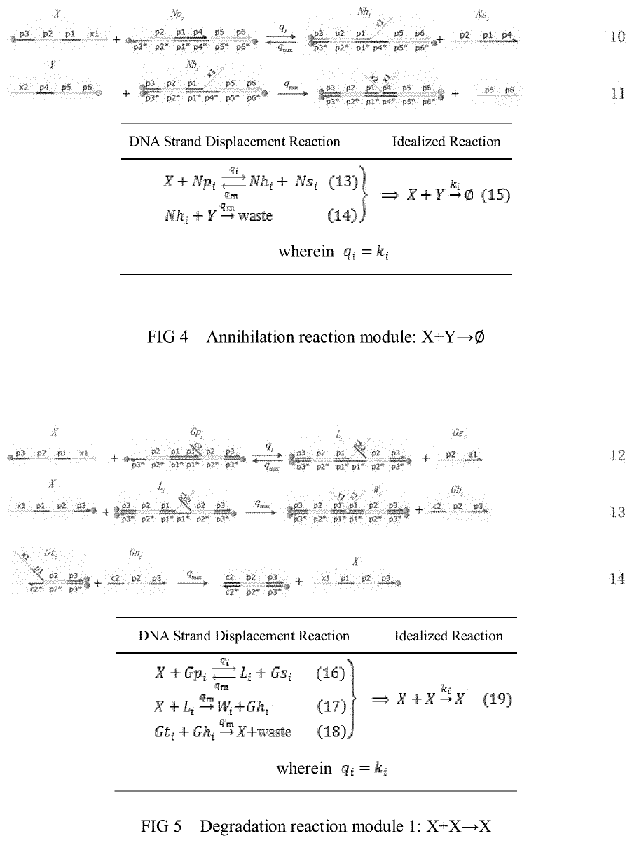

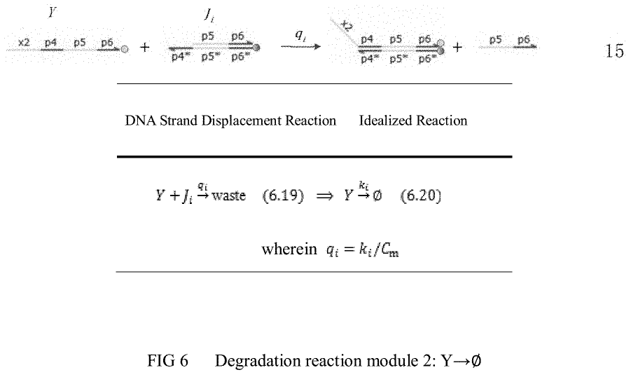

Table 1 Values of parameters of a Rössler chaotic system

Table 2 Correlation coefficient of a “lena” ciphertext image and adjacent pixels thereof

Table 3 Information entropy

Table 4 NPCR and UACI values for encrypted images

### DETAILED DESCRIPTION OF THE INVENTION

The present invention will be described further below with reference to the drawings.

The detailed steps are as follows:

Step 1: determining an idealized reaction network that can describe the Rössler chaotic system as shown in Formula (1).

$$\begin{matrix}{X}_{1} {\to }^{ {k}_{1 }} 2 {X}_{1} & ( 1 a ) \\ 2 {X}_{1} {\to }^{ {k}_{2 }} {X}_{1} & ( 1 b ) \\ {X}_{2} + {X}_{1} {\to }^{ {k}_{3} } 2 {X}_{2} & ( 1 c ) \\ {X}_{2} {\to }^{ {k}_{4} } ∅ & ( 1 d ) \\ {X}_{1} + {X}_{3} {\to }^{ {k}_{5} } ∅ & ( 1 e ) \\ {X}_{3} {\to }^{ {k}_{6 }} 2 {X}_{3} & ( 1 f ) \\ 2 {X}_{3} {\to }^{ {k}_{7} } {X}_{3} & ( 1 g )\end{matrix}$$

Step 2: determining an idealized reaction module and a DNA strand displacement reaction module corresponding thereto, for example, a catalytic reaction module, a degradation reaction module and an annihilation reaction module according to an idealized reaction equation.
Step 3: constructing a DNA strand displacement analog circuit according to the idealized reaction network and the reaction modules.
Step 4: adopting an encryption method. The detailed steps (1)-(5) of the encryption method are as follows:
(1) obtaining a secret key dₖ according to plaintext information, wherein K∈[4,5,6, . . . , ∞];
(2) obtaining chaotic sequences x₁(i), x₂(i) and x₃(i) from the concentration measurement, and obtaining sequences matched with plaintext images from the extended sequences) \hat{X}₁(i) \hat{X}₂(i) and \hat{X}₃(i);
(4) scrambling the color plaintext images at the level of the three components R, G, B in consideration of the relation between the three components R, G, B in a color image;
(5) obtaining scrambling sequences Ψ∈[1,N], Γ={Γ^{r},Γ^{g},Γ^{b}} and Ψ={Ψ^{r},Ψ^{g},Ψ^{b}} by using the extended chaotic sequences;
(6) scrambling the plaintext images to obtain a scrambled image P′;
(7) obtaining a scrambling sequence U by utilizing the extended chaotic sequences;
(8) obtaining a scrambling sequence V by utilizing the extended chaotic sequences; and
(9) performing diffusion operation on the image P′ by utilizing sequences U={Uᵣ,U^{g},U^{b}} and V={V^{r},V^{g},V^{b}} to obtain an encrypted image C={C^{r},C^{g},C^{b}}.
Step 5: adopting a decryption method. The detailed steps (1)-(2) of the decryption method are as follows:
(1) removing the diffusion effect on the encrypted image from the last pixel to the first pixel; and
(2) removing the diffusion effect from the last column (row) to the first column (row) to obtain a plaintext image;

### EXAMPLE 1

The embodiments of the present invention are implemented on the premise of the technical proposal of the present invention, and detailed embodiments and specific operation processes are given, but the scope of protection of the present invention is not limited to the following embodiments.

Step 1: substituting the parameters in table 1 into the DNA strand displacement analog circuit to obtain chaotic sequences x₁(i), x₂(i) and x₃(i).
Step 2: detecting the encryption scheme by using color images of sizes 256×256 including “Lena”, “Pepper” and “Baboon”, wherein s₁=800, s₂=1500.
Step 3: arranging the pixel values of the plaintext image according to the size, then representing the pixel values by p^{r}, p^{g} and p^{b}, and recording the positions of the pixel values respectively by q^{r}×{q₁^{r},q₂^{r}, . . . ,q_{M×N}^{r}}, q^{q}={q₁^{g},q₂^{g}, . . . ,q_{M×N}^{g}} and q^{b}={q₁^{b},q₂^{b}, . . . ,q_{M×N}^{b}, then

$$\begin{matrix}{sum}_{r} = {\sum }_{i = 1}^{M \times N} {p}_{i}^{r} {q}_{i}^{r} & ( 21 ) \\ {sum}_{g} = {\sum }_{i = 1}^{M \times N} {p}_{i}^{g} {q}_{i}^{g} & ( 22 ) \\ {sum}_{b} = {\sum }_{i = 1}^{M \times N} {p}_{i}^{b} {q}_{i}^{b} & ( 23 ) \\ {d}_{k} = mod ( {sum}_{r} + {sum}_{g} + {sum}_{b} , k + 0.1 ) \\ ( 1 \leq k \leq K ) & ( 24 )\end{matrix}$$

wherein, 0<αₖ≤10, K∈[4,5,6, . . . , ∞].

Step 4: extending x₁(i), x₂(i), and x₃(i) into three sets of sequences and \hat{X}₁(i), \hat{X}₂(i) and \hat{X}₃(i) matched with the plaintext images as follows,

$$\begin{matrix}\{ \begin{matrix}{\hat{X}}_{1} ( i ) = {X}_{1} ( m + 1 ) \\ {\hat{X}}_{2} ( i ) = {X}_{2} ( m + 1 ) \\ {\hat{X}}_{3} ( i ) = {X}_{3} ( m + 1 )\end{matrix} \\ 1 \leq i \leq {r}_{2} & ( 25 ) \\ \{ \begin{matrix}{\hat{X}}_{1} ( {r}_{2} + b \times {r}_{1} - j ) = {X}_{1} ( m + 1 + b ) \\ {\hat{X}}_{2} ( {r}_{2} + b \times {r}_{1} - j ) = {X}_{2} ( m + 1 + b ) \\ {\hat{X}}_{3} ( {r}_{2} + b \times {r}_{1} - j ) = {X}_{3} ( m + 1 + b )\end{matrix} & ( 26 ) \\ {r}_{1} = ⌊ M \times N / ( Π - 1 ) ⌋ & ( 27 ) \\ {r}_{2} = mod ( M \times N , Π - 1 ) & ( 28 ) \\ 1 \leq b \leq Π - 1 , 0 \leq β \leq {r}_{1} - 1 & ( 29 )\end{matrix}$$

the first m data are discarded, and the detection time can be obtained by the following equation:

T=(π+m)×3(seconds) (30)

Step 5: scrambling the color plaintext images at the level of the three components R, G, B as follows in consideration of the relation among the three components R, G, B in a color image:

$$\begin{matrix}\{ \begin{matrix}{p}^{r} ( i ) ↔ {p}^{g} ( {q}_{i}^{r} ) & if {\hat{X}}_{1} ( i ) > 10 nM \\ No exchange & if {\hat{X}}_{1} ( i ) \leq 10 nM\end{matrix} & ( 31 ) \\ \{ \begin{matrix}{p}^{g} ( i ) ↔ {p}^{b} ( {q}_{i}^{g} ) & if {\hat{X}}_{2} ( i ) > 10 nM \\ No exchange & if {\hat{X}}_{2} ( i ) \leq 10 nM\end{matrix} & ( 32 ) \\ \{ \begin{matrix}{p}^{b} ( i ) ↔ {p}^{r} ( {q}_{i}^{b} ) & if {\hat{X}}_{3} ( i ) > 10 nM \\ No exchange & if {\hat{X}}_{3} ( i ) \leq 10 nM\end{matrix} & ( 33 )\end{matrix}$$

wherein 1≤θ≤M×N
Step 6: repetitively iterating equations (34) and (35) until 6 one-dimensional arrays are obtained, wherein Γ∈[1,M], Ψ∈[1,N], Γ={Γ^{r},Γ^{g},Γ^{b}} and Ψ={Ψ^{r},Ψ^{g},Ψ^{b}}.

$$\begin{matrix}\{ \begin{matrix}{Γ}^{r} = mod ( floor ( \frac{{\hat{X}}_{1}}{d} \times {10}^{14} ) , M ) + 1 \\ {Γ}^{g} = mod ( floor ( \frac{{\hat{X}}_{1}}{d} \times {10}^{14} ) , M ) + 1 \\ {Γ}^{b} = mod ( floor ( \frac{{\hat{X}}_{3}}{d} \times {10}^{14} ) , M ) + 1\end{matrix} & ( 34 ) \\ \{ \begin{matrix}{Ψ}^{r} = mod ( floor ( \frac{{\hat{X}}_{1}}{d} \times {10}^{14} ) , N ) + 1 \\ {Ψ}^{g} = mod ( floor ( \frac{{\hat{X}}_{2}}{d} \times {10}^{14} ) , N ) + 1 \\ {Ψ}^{b} = mod ( floor ( \frac{{\hat{X}}_{3}}{d} \times {10}^{14} ) , N ) + 1\end{matrix} & ( 35 )\end{matrix}$$

step 7: obtaining a scrambled image P′ according to the following operation:

f(i,j)⇄f(Γ(i+s₁),Ψ(j+s₂)) (36)

Step 8, generating a diffusion sequence U by using the chaotic sequences;

$$\{ \begin{matrix}{U}^{r} = mod ( floor ( \frac{{\hat{X}}_{1}}{d} \times {10}^{14} ) , 256 ) \\ {U}^{g} = mod ( floor ( \frac{{\hat{X}}_{1}}{d} \times {10}^{14} ) , 256 ) \\ {U}^{b} = mod ( floor ( \frac{{\hat{X}}_{3}}{d} \times {10}^{14} ) , 256 )\end{matrix} \qquad ( 37 )$$

Step 9: generating a diffusion sequence V by using the chaotic sequences;

$$\begin{matrix}\{ \begin{matrix}{V}^{r} = 255 & if {\hat{X}}_{1} > 10 nM \\ {V}^{r} = 0 & if {\hat{X}}_{1} \leq 10 nM\end{matrix} & ( 38 ) \\ \{ \begin{matrix}{V}^{g} = 255 & if {\hat{X}}_{2} > 10 nM \\ {V}^{g} = 0 & if {\hat{X}}_{2} \leq 10 nM\end{matrix} & ( 39 ) \\ \{ \begin{matrix}{V}^{b} = 255 & if {\hat{X}}_{3} > 10 nM \\ {V}^{b} = 0 & if {\hat{X}}_{3} \leq 10 nM\end{matrix} & ( 40 )\end{matrix}$$

step 10: performing diffusion operation on the image p′ by using the matrix U={U^{r},U^{g},U^{b}} and V={V^{r},V^{g},V^{b}} to obtain an encrypted image C={C^{r},C^{g},C^{b}}.

$$\begin{matrix}{C}_{1} = {P}_{1}^{′} ⊕ {U}_{i} ⊕ {V}_{1} & ( 41 ) \\ \{ \begin{matrix}{C}_{i}^{r} = {P}_{i}^{′ r} ⊕ {C}_{i - 1}^{r} ⊕ {U}_{i}^{r} ⊕ {V}_{i}^{r} \\ {C}_{i}^{g} = {P}_{i}^{′ g} ⊕ {C}_{i - 1}^{g} ⊕ {U}_{i}^{g} ⊕ {V}_{i}^{g} ⊕ {C}_{i}^{r} \\ {C}_{i}^{b} = {P}_{i}^{′ b} ⊕ {C}_{i - 1}^{b} ⊕ {U}_{i}^{b} ⊕ {V}_{i}^{b} ⊕ {C}_{i}^{r} ⊕ {C}_{i}^{g}\end{matrix} \\ M \times N \geq i > 1 & ( 42 )\end{matrix}$$

step 11: adopting a decryption method. The detailed steps: (1)-(2) of the decryption method are as follows:

(1) removing the diffusion effect on the encrypted image from the last pixel to the first pixel;

$$\begin{matrix}\{ \begin{matrix}{E}_{i}^{r} = {C}_{i}^{r} ⊕ {C}_{i - 1}^{r} ⊕ {U}_{i}^{r} ⊕ {V}_{i}^{r} \\ {E}_{i}^{g} = {C}_{i}^{g} ⊕ {C}_{i - 1}^{g} ⊕ {U}_{i}^{g} ⊕ {V}_{i}^{g} ⊕ {C}_{i}^{r} \\ {E}_{i}^{b} = {C}_{i}^{b} ⊕ {C}_{i - 1}^{b} ⊕ {U}_{i}^{b} ⊕ {V}_{i}^{b} ⊕ {C}_{i}^{r} ⊕ {C}_{i}^{g}\end{matrix} \\ i > 1 & ( 43 ) \\ {E}_{1} = {C}_{1} ⊕ {U}_{1} ⊕ {V}_{1} & ( 44 )\end{matrix}$$

(2) removing the scrambling effect from the last column (row) to the first column (row) to obtain a plaintext image;

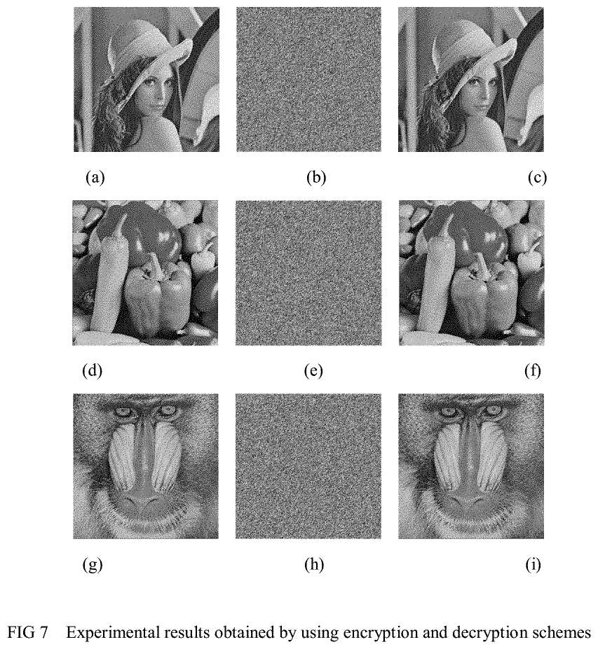

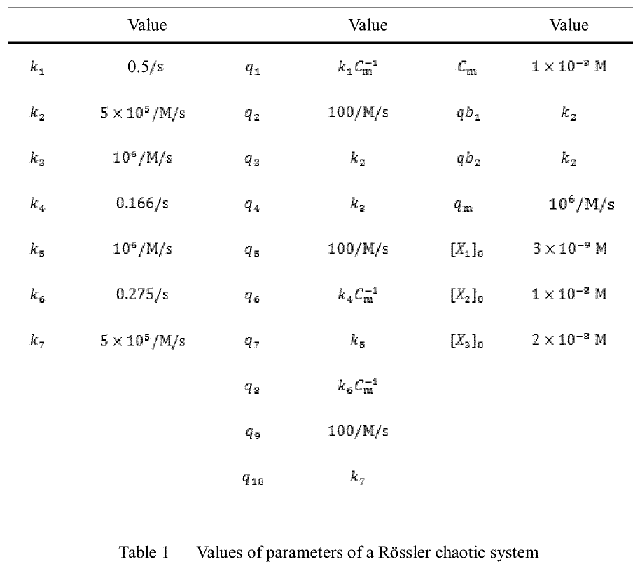

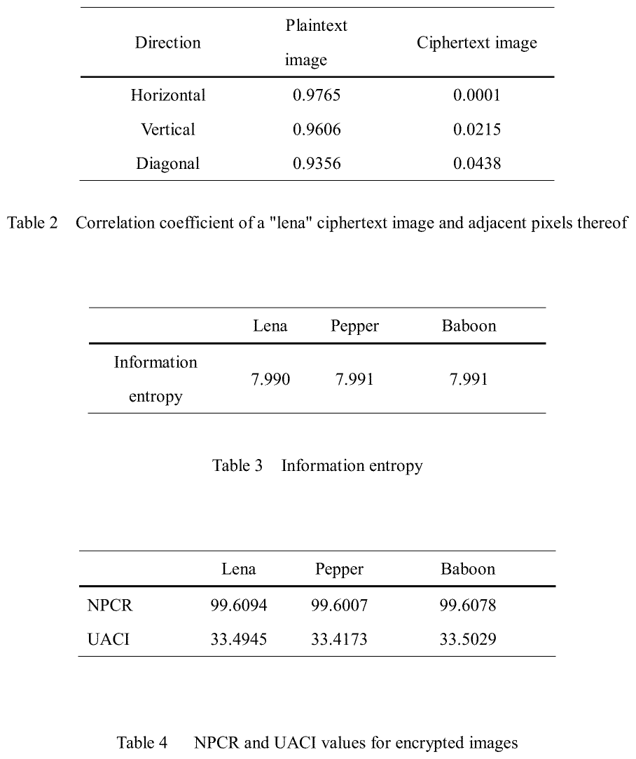

The effects of encryption and decryption of “Lena”, “Pepper” and “Babook” images are shown in FIG. 7, and the indices of the encrypted images are shown in tables 2-4.

The above is only the specific embodiment of the present invention, but the protection scope of the present invention is not limited thereto, and any changes or substitutions without creative efforts shall fall within the protection scope of the present invention. Therefore, the claimed protection extent of the invention shall be determined with reference to the appended claims.

1. A color image encryption method based on a DNA strand displacement analog circuit, which essentially combines a DNA strand displacement technology with an image chaotic encryption method by utilizing the compilability of a DNA strand sequence, and the method comprises the following specific steps:
step 1: determining an idealized reaction network that can describe a Rössler chaotic system;
step 2: determining an idealized reaction module and a DNA strand displacement reaction module corresponding thereto according to an idealized reaction equation;
step 3: constructing a DNA strand displacement analog circuit according to the idealized reaction network and the reaction module;
step 4: adopting an encryption method, and the detailed steps (1)-(5) of the encryption method are as follows:
(1) generating a secret key by using the plaintext image;
(2) setting the measurement accuracy of the concentration of a DNA strand as 0.1 nM, wherein the measurement is performed every 3 seconds;
(3) extending the measured data to obtain a new chaotic sequence;
(4) scrambling the color plaintext image at the level of the three components of R, G, B in consideration of the relation between the three components of R, G, B in a color image;
(5) performing scrambling at a pixel level by utilizing the extended chaotic sequence;
(6) generating a scrambling sequence U by using the chaotic sequence;
(7) generating a scrambling sequence V by using the chaotic sequence; and
(8) performing diffusion processing on the scrambled image;
step 5: adopting a decryption method, which comprises: removing the diffusion effect on the encrypted image according to the inverse process of the encryption method to obtain a plaintext image;

2. The color image encryption technology based on a DNA strand displacement analog circuit according to claim 1, wherein the color image encryption is performed by using the DNA strand displacement chaotic analog circuit for the first time.
